(19) United States
(12) Patent Application Publication
(10) Pub. No.: US 2012/0149697 A1
(43) Pub. Date: Jun. 14, 2012

(54) HUMIC SUBSTANCES AND THERAPEUTIC USES THEREOF

(75) Inventors: Giuseppe Antonio Legname, Trieste (IT); Liviana Leita, Moruzzo (UD) (IT); Paolo Sequi, Pietrasanta (LU) (IT)

(73) Assignee: SCUOLA INTERNAZIONALE SUPERIORE DI STUDI AVANZATI, Trieste (IT)

(21) Appl. No.: 13/384,127

(22) PCT Filed: Jul. 13, 2010
(86) PCT No.: PCT/IB2010/053204

§ 371 Date: Mar. 2, 2012

Related U.S. Application Data
(60) Provisional application No. 61/226,026, filed on Jul. 16, 2009.

(51) Int. Cl. A61K 31/538; A61P 25/16; A61K 31/194; A61P 25/28; C07D 413/14; C07C 65/40

(52) U.S. Cl. 514/229.8; 544/102; 562/462; 514/569

(57) ABSTRACT

The present invention relates to the medical field, in particular to the use of natural organic polyanions, i.e. humic substances, HSs, in the treatment of neurodegenerative diseases, such as Prion disease, Alzheimer's and Parkinson's disease.

### FIELD OF INVENTION

The present invention relates to the medical field, in particular to the use of natural organic polyanions, i.e. humic substances, HSs, in the treatment of neurodegenerative diseases, such as Prion disease, Alzheimer's and Parkinson's disease.

The interaction of the recombinant prion protein with a class of refractory natural organic polyanions, humic substances (HSs), polydisperse mixtures of polyphenolpolycarboxylic acids, possessing self associating and colloidal proprieties is demonstrated in the present invention.

### STATE OF THE ART

Prion diseases (or TSE, Transmissible Spongiform Encephalopathies), Alzheimer's disease (AD), Parkinson's disease, amyotrophic lateral sclerosis, and Huntington's disease are all neurodegenerative diseases, the incidences of which increase with age (Lilienfield, 1993). These age-dependent diseases are becoming an increasingly serious public health problem in developed countries, where modern medicine as well as improved hygiene and lifestyles contribute to extending the average life span.

The age dependence of AD is dramatic: incidence is about 1% at age 60 but approaches 30% by age 85 (Katzman 1986; Evans et al. 1989; Hebert et al. 2003; Spencer et al. 2007). Prions are infectious pathogens causing transmission of the disease collectively known as the transmissible spongiform encephalopathies (TSE) thus causing fatal neurodegenerative disorders in different mammalian species, e.g. scrapie in sheep, bovine spongiform encephalopathy (BSE) in cattle, chronic wasting disease (CWD) in mule deer, elk, and moose (cervids), and Creutzfeldt-Jakob disease (CJD/vCJD) in humans. Unlike Alzheimer's disease, prion diseases are relatively rare. Each year only approximately 300 people in the USA and approximately 100 people in the UK succumb to various forms of prion diseases (Sigurdsson and Wisniewski, 2005). Nevertheless, these disorders have received great scientific and public interest due to the fact that they can be transmissible among humans and in certain conditions from animals to humans (i.e. the BSE, bovine spongiform encephalopathy). Therefore, the spread of cervids prion diseases (CWD) across North America could put a large human population at risk of prion infection.

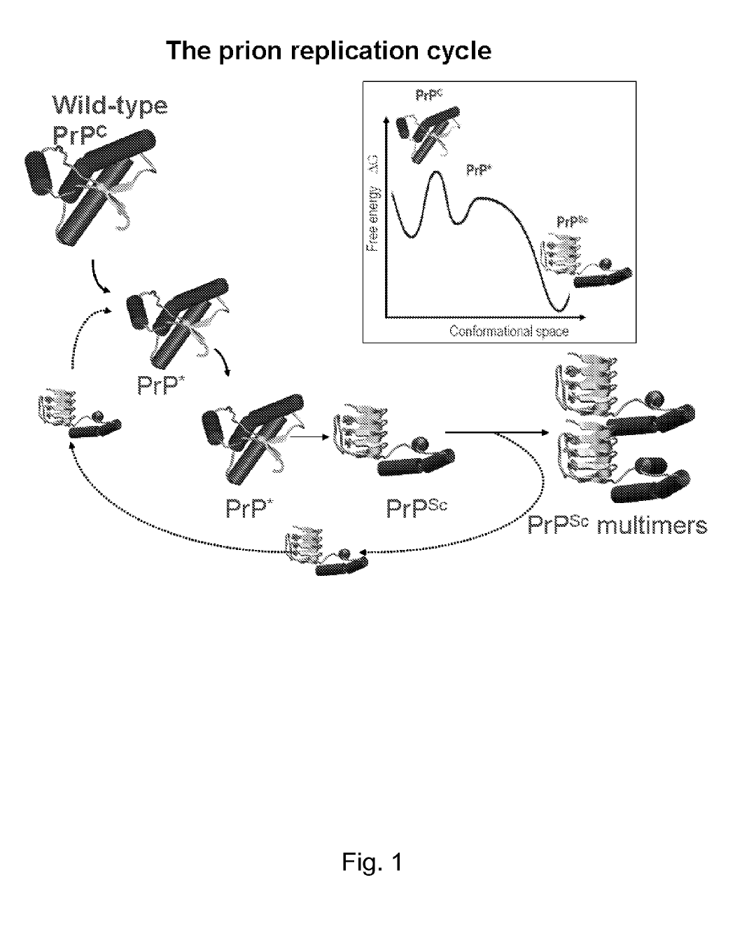

Prion diseases are a rare and fascinating group of neuropathies characterized by (i) a spongiform neurodegeneration of the brain, (ii) the amyloid fibrils deposition composed by the abnormal, misfolded form of the cellular prion protein (PrP^{C}) and (iii) a unique etiology that could be sporadic, inherited and iatrogenic. The prion is devoid of nucleic acid and it is formed solely by the pathogenic form of the cellular prion protein named PrP^{Sc}, where Sc means scrapie, the prototypical prion disease of sheeps and goats. As we can see in FIG. 1, the normal form (PrP^{C}) is converted in the abnormal one (PrP^{Sc}) by a not well identified process of conversion from the α-helix motives into β-sheet secondary structures (Prusiner, 1998).

In other neurodegenerative diseases, such as Alzheimer's (AD) or Parkinson's (PD) disease, the pathological mechanism is similar to TSE: a conformational change of normally expressed proteins, i.e. amyloid-β in AD or synuclein in PD (Wisniewski and Sigurdsson, 2007). Neurological symptoms in AD, PD and TSE are directly related to loss of neurons and synaptic connections.

Over the past 10 years there have been various efforts to discover drugs and compounds effective in prion disease. These include: porphyrins (Priola et al, 2000), Congo red and its derivatives (Caspi et al, 1998), acridine and phenothiazine derivatives (Doh-Ura et al, 2000), heparan sulfate (Adjou et al, 2003), aminoglycan and polyamines (Supattapone et al, 2003). Simultaneously, various technological developments have been reported including structure-based drug design followed by the structure—activity relationship study (Kuwata et al, 2007), small interfering RNA (Daude et al, 2003), library screening (Kocisko et al, 2003), high-throughput screening (Bertsch et al, 2005), immunoterapy approach (Campana et al, 2009), and so on.

These prion antagonists can be targeted towards the selective binding of PrP^{C} or PrP^{Sc} and/or to the process of conversion. However, most of these molecules were found to be toxic or ineffective for the infected host.

The present invention evidences the interaction of the two main categories of soil compounds, humic substances, with the prion protein. Here, the authors report for the first time the anti-prion activity exerted by two soil compounds: the humic and fulvic acids. In particular, they report that non cytotoxic concentrations of natural organic polyanions, humic (HA) and fulvic (FA) substances, can rapidly eliminate PrP^{Sc} from chronically infected ScGT1 cells.

Humic substances are a ubiquitous reservoir of carbon in soil and natural waters representing the bulk of organic matter of soil, peat, lignites, brown coals, sewage, natural waters and their sediments. Being the decay products of the total biota in the environment, they are highly refractory. They are formed through aerobic and anaerobic decomposition of plant and animal detritus, as well as secondary microbial synthesis. Their chemical structure is mainly built up by heteroatomic functionalities including phenols and other alcohols, ketones/quinones, aldehydes, carboxylic acids, amino- and nitro-groups, and sulfur containing entities such as mercaptans, sulfates, and sulfonates. However, the term ‘humic substances’ is used in a generic sense to distinguish the naturally occurring material from the products of chemical extractions named humin, humic acids (HAs,) and fulvic acids (FAs), which are defined “operationally” by their solubility in alkali or acid solutions. Humic acids are soluble in alkaline solution, fulvic acids are soluble in both alkaline and acidic solution, while humin represents the insoluble residue. It is possible to envisage a general molecular configuration of the chemical structure of HSs, HAs and FAs in particular, so that we speak of hypothetical model of basic block-structures like those reported below (see also Stevenson, 1994).

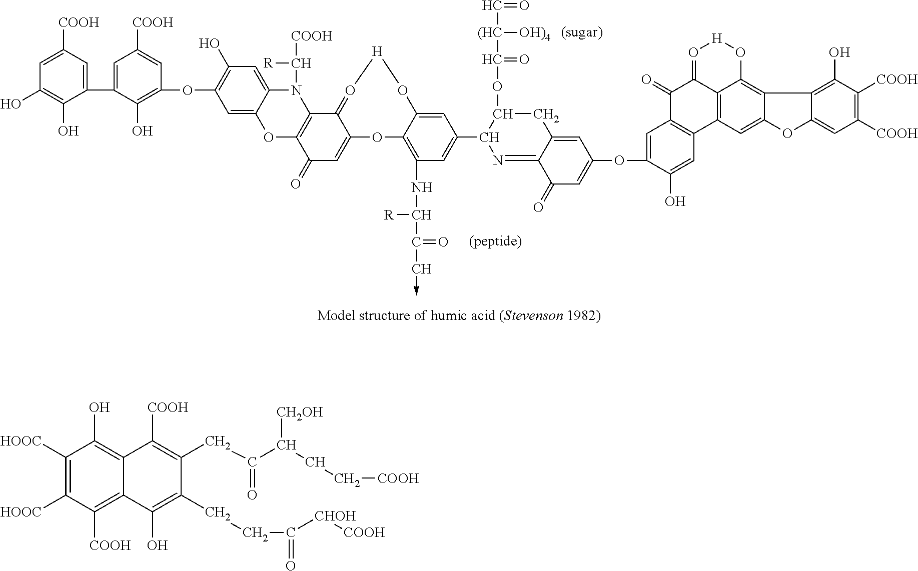

Humic and fulvic acids (HAs, FAs) are natural carbon-rich polydisperse polyanionic (at natural conditions) biopolymers, whose multiple properties seem to be purpose-built for many life-sustaining functions from agriculture (e.g., field fertilization apart, humates can also be used in animal husbandry for growth stimulation purposes) to industry (es. production of fertilizers) and biomedicine (e.g., co-products in cosmetics, antivirals, drugs for the stimulation of the immune system, detoxifying properties) (Pena-Mendez, 2005; Schiller et al. 1979, Zeck-Knapp et al. 1991; Schneider et al. 1996; Schermer et al. 1998). However, while these two classes of compounds share many structural features, including an abundance of carboxy, hydroxy, phenolic, and ketonic groups, FAs have lower molecular weight, higher functional group density, and higher acidity than HAs. The majority of HAs' functional groups include carboxylic, phenolic, hydroxyl, carbonyl, amine, amide and aliphatic moieties, among others.

One of the most significant properties of HAs and FAs or/and HA/FA-like substances is their ability to interact with xenobiotics to form complexes of different solubility and chemical and biochemical stability. Due to this polyfunctionality, HAs FAs therefore represent a strongly pH dependent reservoir of electron donors/acceptors, which could hypothetically contribute to reduction-oxidation of several inorganic and organic agents (Pacheco et al 2003). They are able to complex heavy metals (Lubal et al., 1998; Kurk and Choppin, 2000, Borges et al., 2005; Campitelli et al., 2006; Lubal et al., 1998), radio-nuclides (Lubal et al., 2000; Pacheco and Havel, 2001), inorganic anions (Leita et al., 2001, 2009), halogens (Lee et al., 2001; Myneni, 2002), organic acids (Cozzolino et al., 2001), aromatic compounds (Schulten et al., 2001; Narri and Kim, 2002), pesticides and herbicides (Chien and Bleam, 1997; De Paolis and Kukkonen, 1997; Schmitt et al., 1997; Fang et al., 1998; Shigemasa and Mamoru, 1999; Gevao et al., 2000; Klaus et al., 2000), viruses and proteins (Klocking et al., 1972, 1991, Schols et al., 1991, Loya et al., 1993).

In addition, chemically HAs and FAs behave as supramolecules (Steed and Atwood, 2000) which are able to polymerize and aggregate (Fetsch et al., 1998), form micelles (Guetzloff and Rice, 1994) and might also form supramolecular ensembles with other compounds.

In the present invention, it was surprisingly found that natural organic polyanions, humic (HA) and fulvic (FA) substances, remove prion infectivity from living cells that were chronically infected. The authors describe that HA and FA could purge mouse scrapie-infected hypothalamic (ScGT1) cells of PrP^{Sc} (the disease-causing isoform of the prion protein) in a dose dependent manner without affecting cell viability. Furthermore, they confirmed that this inhibition occurs not only in vivo but also in vitro.

To the authors' knowledge, this is the first class of natural soil compounds shown to abate prion infection. The present invention clearly establishes the potential of HSs to promote the elimination of detectable PrP^{Sc}.

One possible mechanism is that HSs could act as a chaperon compound, the direct binding with PrP^{C} blocking the conversion reaction from PrP^{C} to PrP^{Sc}.

Such hypothesis has found an astonishing merit in developing effective therapeutics for one or more of the common degenerative illnesses, including Alzheimer's disease, Parkinson's disease, amyotrophic lateral sclerosis, frontotemporal dementia, adult onset diabetes mellitus, and the amyloidoses. For that reason HSs could be efficacious in a variety of inherited disorders where the accumulation of abnormal proteins is a hallmark of the illness.

To the authors' knowledge, this is the first class of natural soil compounds shown to cure an established prion infection. The main benefit produced by the invention is to dispose of a compound able to inhibit the process of conversion from the α-helix (PrP^{C}) motives into β-sheet secondary structures (PrP^{Sc}).

As mentioned above, the same pathological mechanism concerns some neurodegenerative diseases, such as the Alzheimer's (AD) or Parkinson's (PD) ones. These diseases, in fact, cause a conformational change of the normally expressed proteins, i.e. amyloid-β in AD or synuclein in PD.

Learning about the mechanisms responsible for the delayed onset of prion diseases may eventually help decipher the age-dependent onset of several other degenerative disorders of the central nervous system (CNS).

Treatments capable of arresting or at least effectively modifying the course of disease do not yet exist for either one of these diseases. Thus there is a need for such treatments.

### SUMMARY OF INVENTION

It is therefore the object of the present invention a humic substance for use in the treatment of a neurodegenerative disease.

It is another object of the invention a composition comprising a humic substance and appropriated diluents or excipients for use in the treatment of a neurodegenerative disease.

It is another object of the invention a method for the treatment of a neurodegenerative disease comprising administering to a subject in need thereof a humic substance.

Preferably, the neurodegenerative disease is selected from the group of: prion disease, Alzheimer's disease, Parkinson's disease, amyotrophic lateral sclerosis or Huntington's disease. Still preferably, the prion disease is selected from the group of: scrapie, bovine spongiform encephalopathy, chronic wasting disease or Creutzfeldt-Jakob disease.

Yet preferably, the humic substance is humic acid, fulvic acid or a mixture thereof.

The invention will be now described by non limiting examples referring to the following figures:

FIG. 1: The prion replication cycle model. According to the “protein-only hypothesis” by Stanley Prusiner, the conversion occurs without the need of any DNA information. During the disease, the normal form (PrP^{C}) is converted in the abnormal one (PrP^{Sc}) passing through a less stable intermediate conformer (PrP*) by a not well identified process of conversion from the α-helix motives into β-sheet secondary structures. PrP^{C} and PrP^{Sc} are characterized by the same chemical properties, but different secondary structures and physiochemical properties. PrP^{Sc}, unlike PrP^{C}, gives rise to highly ordered protein aggregate, fibrils or oligomers (PrP^{Sc} multimers). PrP^{Sc} can bind PrP^{C} which, in turn, is converted in the abnormal form too. In the upper right panel, the Gibbs free energy (or Gibbs function) is displayed energy as a function of the conformational space explaining the different Energy state from PrP^{C} to PrP^{Sc} (modified from Cohen and Prusiner, 1998).

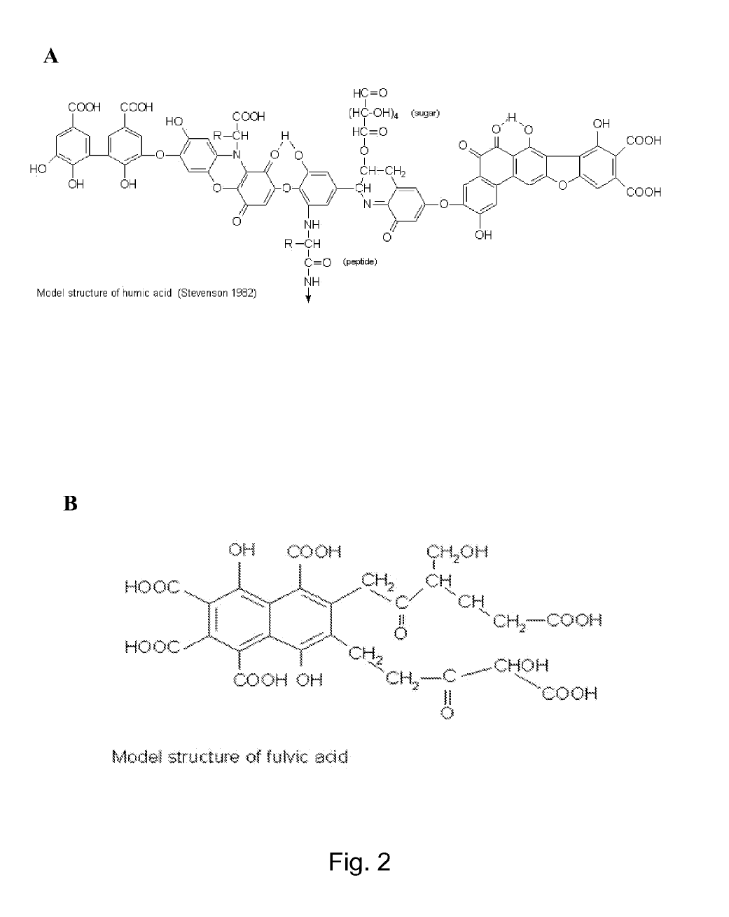

FIG. 2: Model structure of humic (A) and fulvic (B) acid (see also Stevenson, 1994)

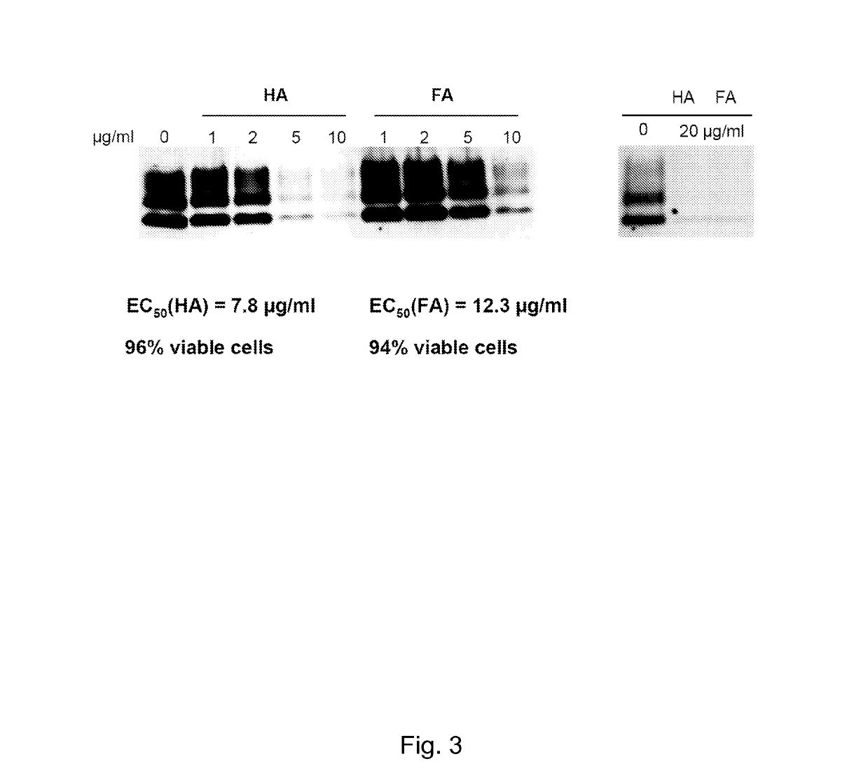

FIG. 3: Humic substances induce clearance of pre-existing PrP^{Sc}. ScGT1 cells are chronically infected by PrP^{Sc}. Western blot showing the dose dependent removal of PrP^{Sc} from ScGT1 cells. These compounds have a half maximal effective concentration (EC₅₀) of 7.8 μg/mL and 12.3 μg/mL for HA and FA, respectively. 96% and 94% of the cells remained viable after treatment with a half maximal effective concentrations of HA or FA, respectively.

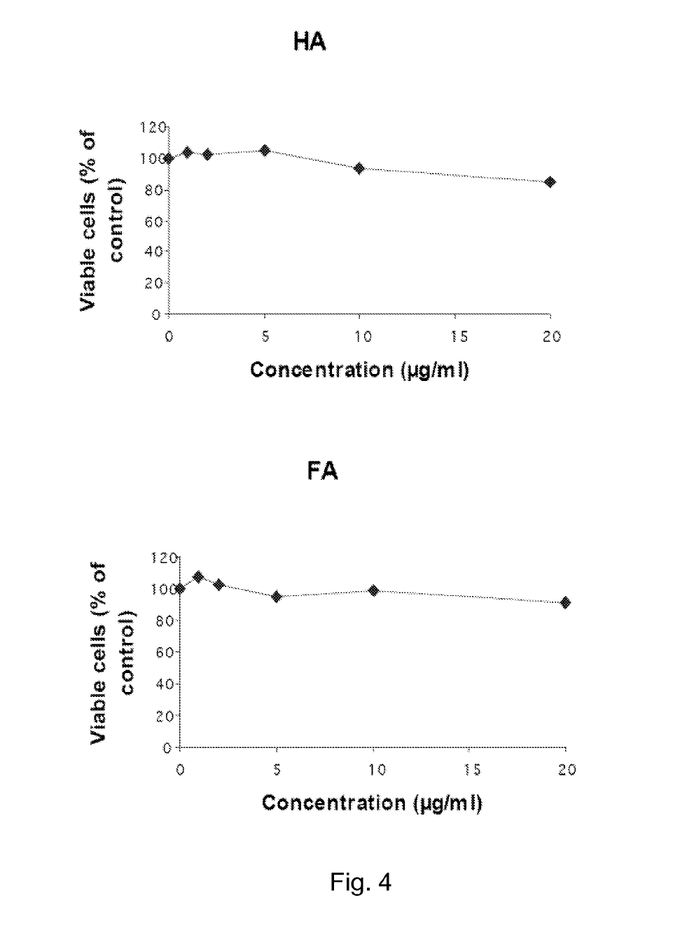

FIG. 4: Humic substances induce clearance of pre-existing PrP^{Sc}. Cell viability test to evaluate the cyto-tossicity effect of HA and FA on ScGT1 cells. Cell remain viable in the presence of different concentration of HA and FA.

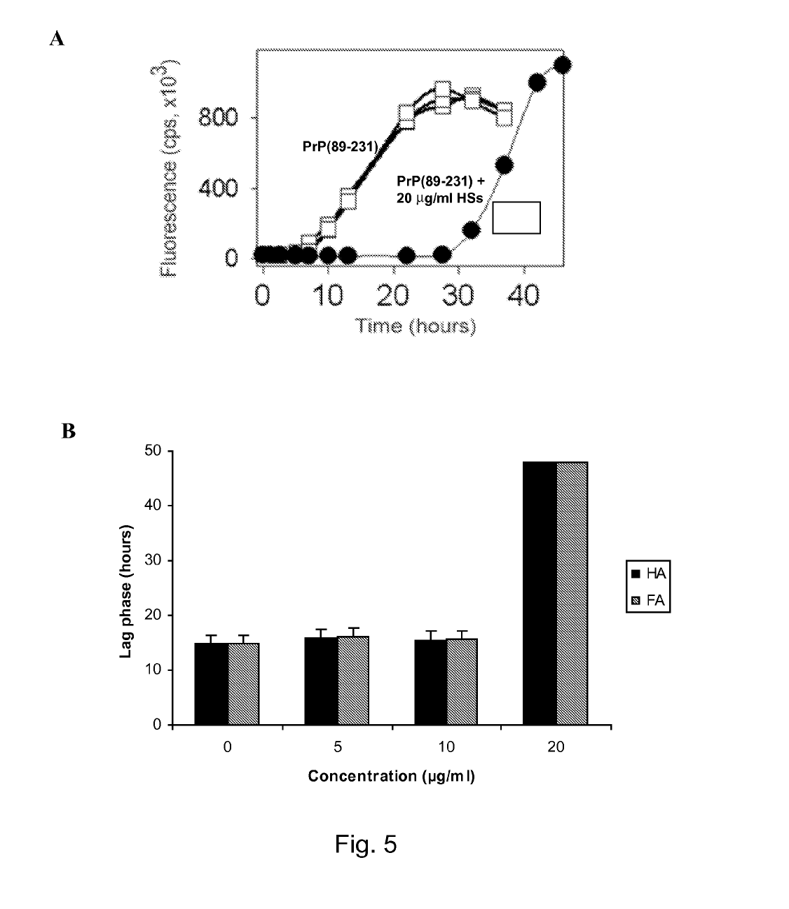

FIG. 5: Humic substances induce inhibition of fibrils formation using recPrP (MoPrP89-231). A) Thioflavin T (ThT) assay: the lag phase of MoPrP(89-231) with () and without (□) the presence of 20 μg/mL Humic Substances (HA or FA) can be observed. The graph represents typical ThT fibrillation assay performed in the presence of HA. Similar results were obtained in the presence of FA. In B) effect in lag phase duration after addition of different amount of HA (black) and FA (grey).

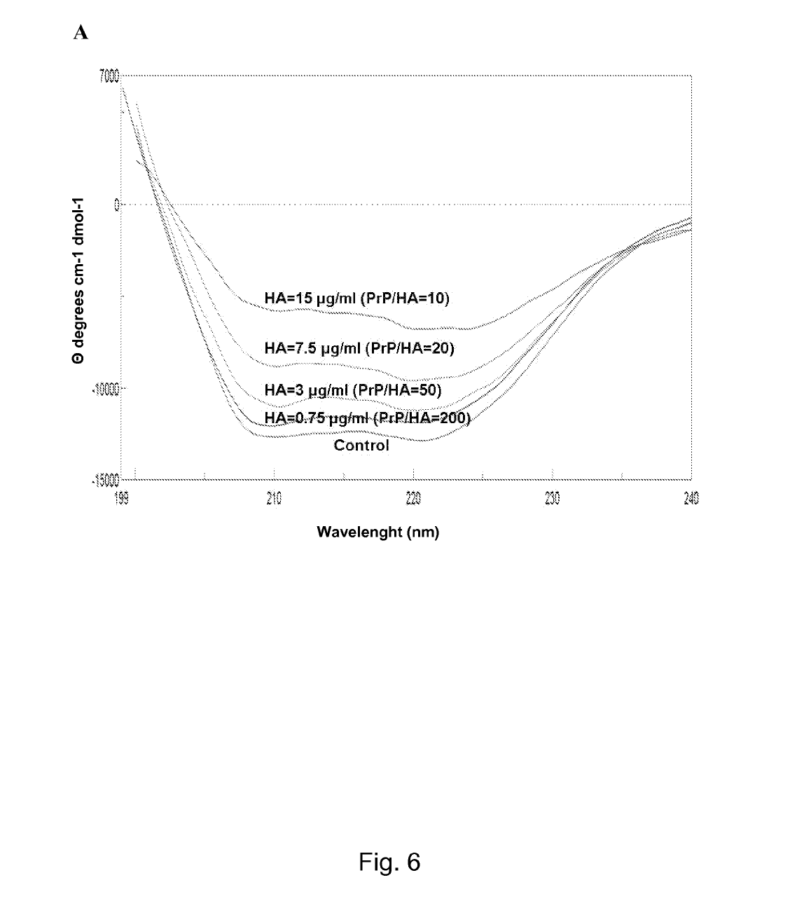

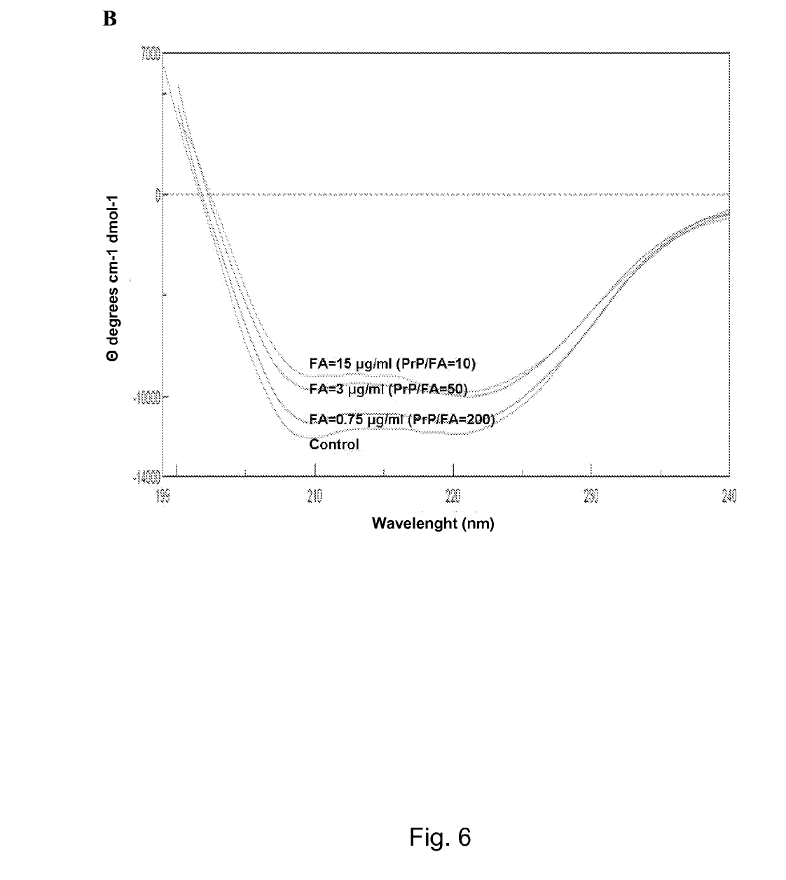

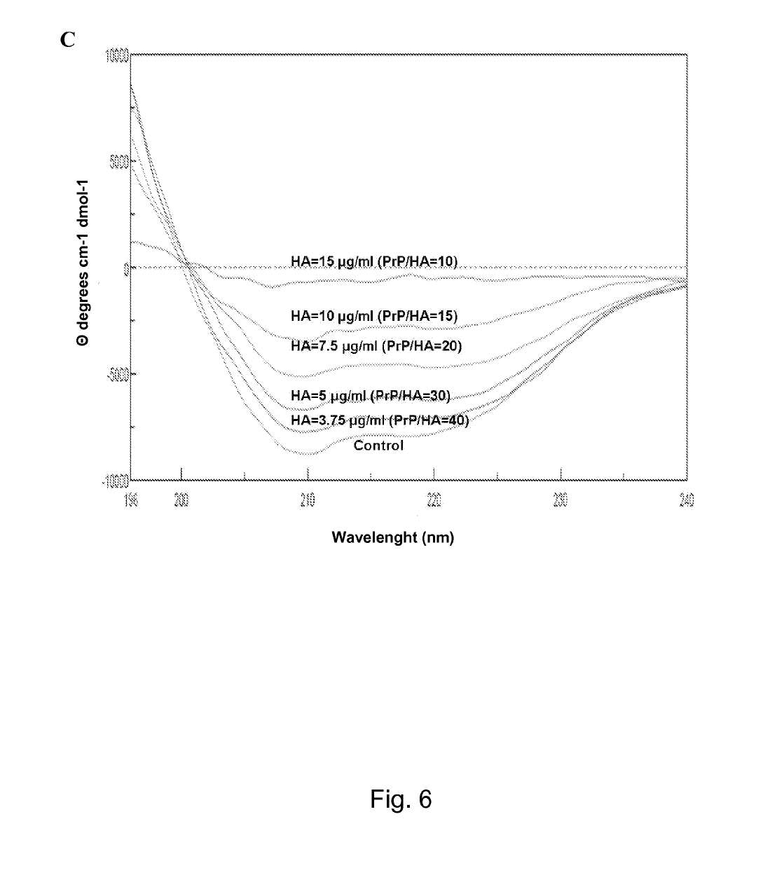

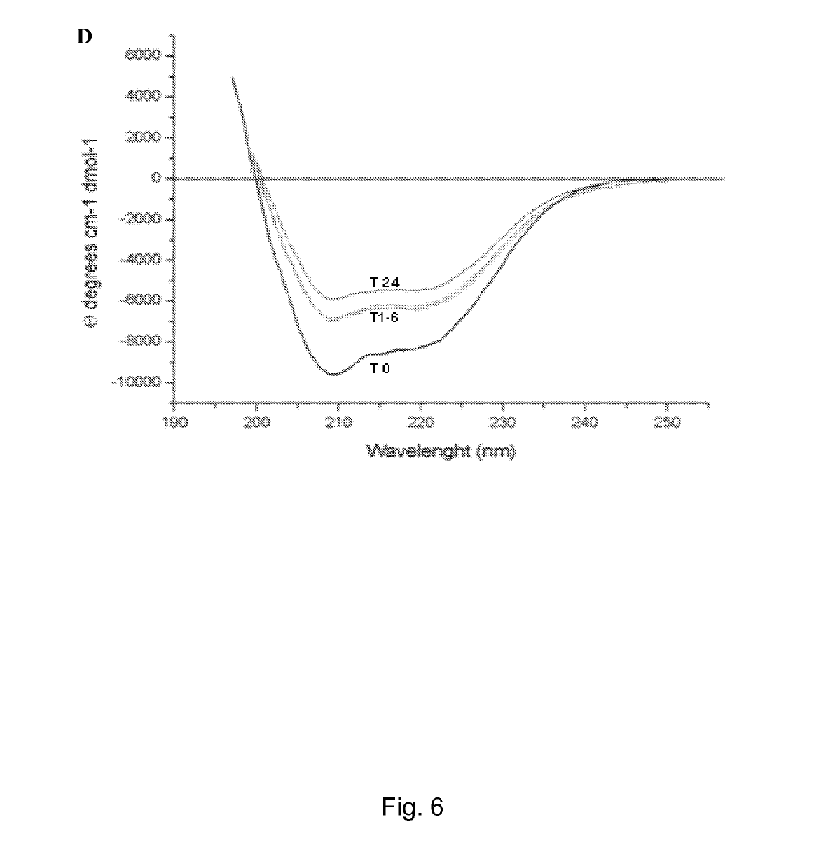

FIG. 6: The addition of 0.75, 3, 7.5, 15 μg/mL of HA (A) or FA (B) to the PrP protein (MoPrP(89-231) provokes a decrease in negative ellipticity of MoPrP(89-231). The same phenomenon is observed in MoPrP(23-231) after the addition of HA (C). D) Far UV-CD time dependent transition of MoPrP(23-231) (0.15 mg/mL) in the presence of Humic Acid (3.75 μg/mL).

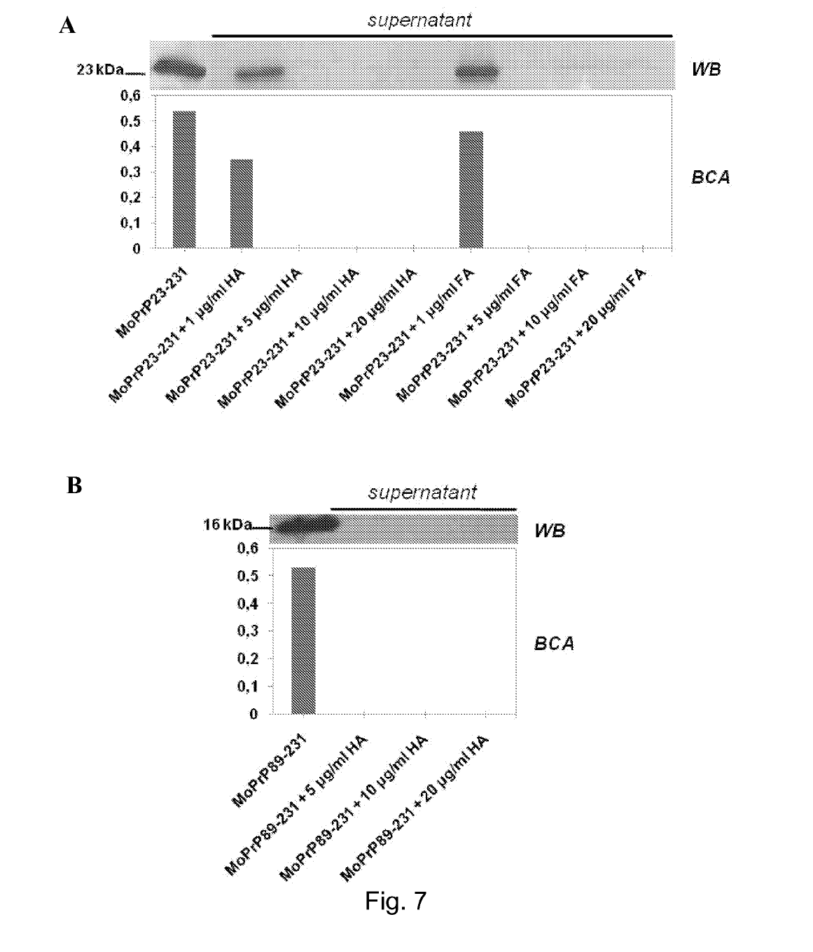

FIG. 7: Adsorption of 20 μg of MoPrP(23-231) (A) and MoPrP(89-231) (B) in the presence of 1 μg/mL to 20 μg/mL of HA and FA. No PrP protein was detected in supernatant solutions after incubation of PrP proteins with HA or FA (5-20 μg/mL), as demonstrated by Western-blotting (WB) and BCA (bicinchoninic acid) protein assay (Pierce).

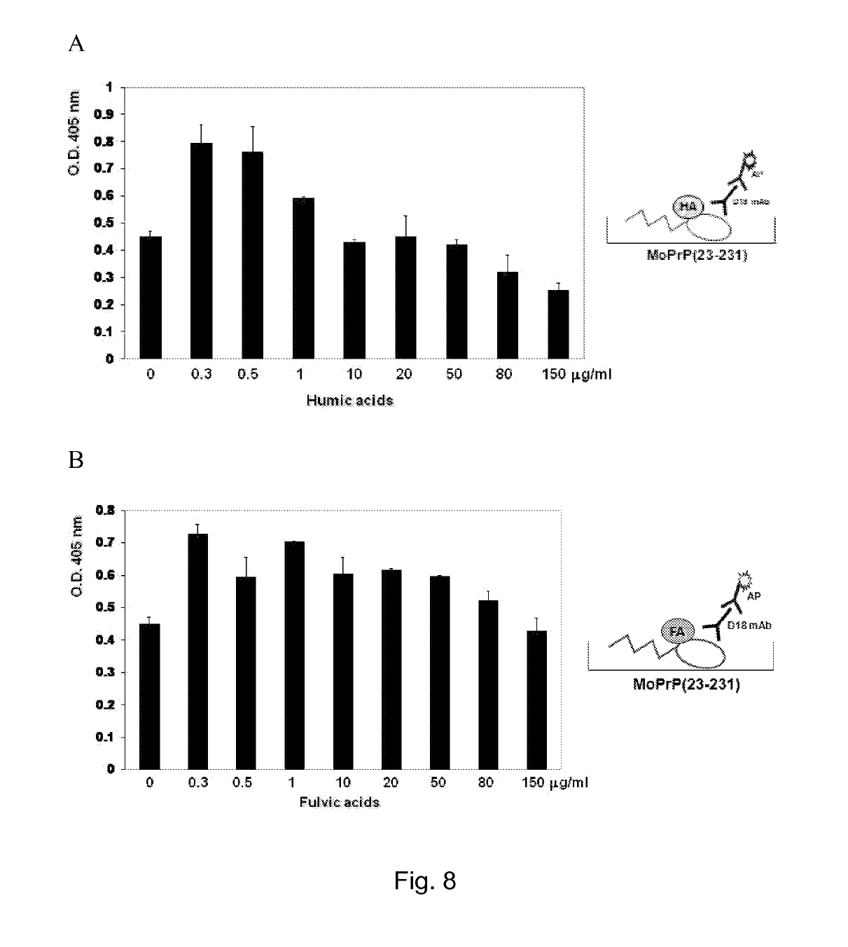

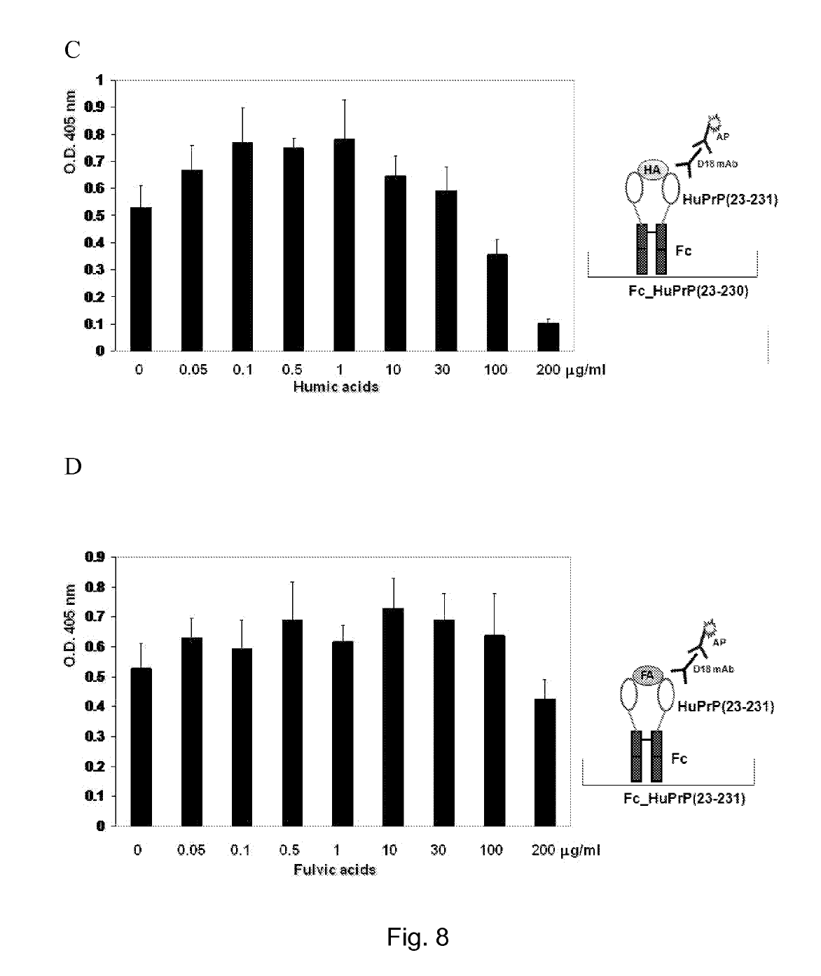

FIG. 8: Competitive ELISA assay using MoPrP(23-231) and HA (A) and FA (B). In (C) and (D) the competitive ELISA using Fc_HuPrP(23-230) and HA and FA, respectively. Coating has been performed using 1 μg for both proteins. Incubation of PrP-coated wells (either with MoPrP(23-231) or Fc_HuPrP(23-230)) with HA at concentrations HA≧100 μg/mL led to a significant decrease in absorbance due to the competitive effect between D18 antibody and HA for coated PrP proteins.

To test the binding propensity of HA and FA on another prion protein the authors used the Fc_HuPrP(23-230): this protein contains a Fc fragment linked to the N-terminal part of the PrP and it has the advantage to expose better the protein into the ELISA well.

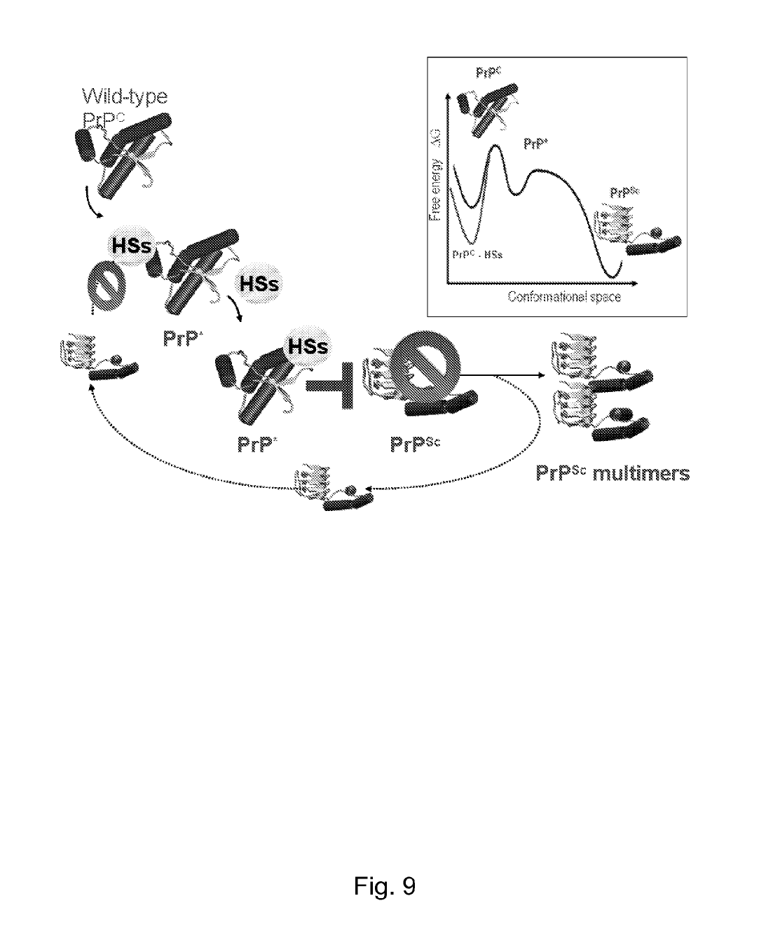

FIG. 9: Model of a possible mechanism of action of HSs during the conversion from PrP^{C} to PrP^{Sc}. The direct binding of HSs with PrP^{C} could block the conversion reaction to the pathogenic form, aging as a chaperon like compound. HSs could stabilize the PrP^{C} conformation and increase the free energy necessary for the aberrant transition (upper right panel). (Modified from Cohen and Prusiner, 1998).

### DETAILED DESCRIPTION OF THE INVENTION

The authors have extracted humic substances from agricultural soil and have separated and purified humic or fulvic acids in accordance with the protocol indicated in Example 5.

### Example 1

To determine whether HA and FA substances can cure ScGT1 cells of scrapie infection, the authors exposed the cells to increasing concentration of HA and FA.

Materials and methods—After exposure for 1 week to an increasing concentration of HA or FA (1, 2, 5, 10 and 20 μg/mL), ScGT1 cells (Schatzl et al., 1997) were harvested and lysis was performed by Lysis Buffer (0.25-1 mL 20 mM Tris, pH 8.0, containing 100 mM NaCl, 0.5% Nonidet P-40, and 0.5% sodium deoxycholate) to obtain a total protein concentration of 0.1 mg/mL measured by the bicinchoninic acid assay (Pierce). Subsequently samples were incubated with 2 μg of proteinase K (Boehringer Mannheim) for 1 h at 37° C. Digested samples were then mixed with equal volumes of 2×SDS sample buffer. All samples were boiled for 10 min prior to SDS-polyacrylamide gel electrophoresis. After electrophoresis, Western blotting was performed. Blocked membranes were incubated with primary D18 monoclonal antibody (to detect mouse PrP) at 1:1,000 dilution in PBST overnight at 4° C. After incubation with primary antibody, membranes were washed and incubated with horseradish peroxidase-labeled secondary antibody (Amersham Life Sciences), diluted 1:5,000 in PBST for 45 min at RT, and washed again. After chemiluminescent development with enhanced chemiluminescence (ECL) reagent (Amersham) for 1 min, blots were exposed to ECL Hypermax film (Amersham). Since PrP^{Sc} is proteinase K resistant, this is a rapid diagnostic test to evaluate the presence of prion in infected cells.

Results—After 1 week, the treatment with HA and FA compounds caused the disappearance of PrP^{Sc} from ScGT1 cells in a dose dependent manner without affecting cell viability (FIG. 3). These compounds have a half maximal effective concentration (EC₅₀) of 7.8 μg/mL and 12.3 μg/mL for HA and FA, respectively. From these data, it is clear that the most potent compounds with respect to eliminating PrP^{Sc} were humic acids. The concentration of humic substances required to eliminate >95% of preexisting PrP^{Sc} was 20 μg/mL for both compounds. The potency of both HSs compounds in eliminating PrP^{Sc} seems dependent on their molecular weight. In fact, HA and FA have a molecular weight of 4,000 Da and 1,500 Da, respectively.

### Example 2

The preceding results demonstrate the potent ability of HSs compounds to clear PrP^{Sc} from ScGT1 cells. To explore whether these compounds could be used as a potential therapeutic for treatment of prion disease, we tested whether they were cytotoxic for ScGT1 cells, using as criteria cell growth, morphology, and viability as measured by trypan blue staining None of the compounds was cytotoxic to ScGT1 cells after exposure for 1 wk at concentrations up to 20 μg/mL (FIG. 4).

### Example 3

Encouraged by their success in reversing the accumulation of PrP^{Sc} in ScGT1 cells under non-cytotoxic conditions, the authors tested the anti-prion activity of HSs substances using an in vitro amyloid conversion assay for prions. This test represents a useful tool to simulate the aggregation kinetics of the prion protein. The presence of drug-compounds binding PrP^{C} could have an effect on the kinetic of fibrils formation. The lag phase corresponds to the time prior the fibrils formation. Stronger is the effect of a drug longer is the lag phase.

In this experiment, the authors observed that HSs compounds strongly inhibit the aggregation propensity of MoPrP(89-231). In particular, they observed that the lag phase of MoPrP(89-231) is longer in the presence of 20 μg/mL of either HA or FA.

Materials and Methods—To monitor the fibril formation the authors performed the Thioflavin T (ThT) assay. ThT fluorescence has been monitored at an emission wavelength of 485 nm and an excitation wavelength of 450 nm. During the time course of amyloid formation, a solution of ThT, 20-fold more concentrated than the final protein concentration, in phosphate buffered saline has been added to aliquots of 10 μg recombinant PrP at room temperature, 25° C. and 37° C. In situ, fluorescence will be monitored in a 96-well fluorescence plate reader (450 nm excitation and 485 nm emission). ThT fluorescence intensity has been read automatically every minute with shaking between measurements. For the screening of HSs compounds different concentration of HA and FA (5-10-20 μg/mL) has been added to the MoPrP solutions (50 μg/mL). For this experiment the authors used two types of recombinant Mouse Prion Protein (Accession number: NP_{—}035300): one including residues from 89 to 231 (MoPrP(89-231)) and the other including residues from 23-231 (MoPrP(23-231)). The first one is the canonical PrP fragment found in amyloid plaque during prion disease, whereas the second one is the mature physiological prion protein.

Results—Anti prion propensity of HSs has been evaluated considering the time required to the recPrP solutions to form fibrils. The time prior to the fibrilization is called lag phase. In FIG. 5A) we can observe the Thioflavin T (ThT) assay with the lag phase of MoPrP(89-231) with () and without (□) the presence of 20 μg/mL Humic Substances (HA or FA). In the presence of a concentration of HSs≧20 μg/mL we observed a significant longer lag phase in comparison with the control (FIG. 5B).

This test supports our findings that HA and FA act as anti-prion agent both in vivo and in vitro.

### Example 4

To start to elucidate the mechanism of action of HSs on the PrP, the authors investigate the effect of HA and FA on the secondary structure of recMoPrP(89-231) and recMoPrP(23-231) using: (i) Far-UV Circular Dichroism (CD), (ii) adsorption assay using Western blot and BCA (Pierce) analysis of the supernatant solutions after ultracentrifugation, (iii) ELISA.

In the absence of HA or FA, the spectra MoPrP(89-231) and MoPrP(23-231) have a double minimum at 222 and 208 nm, characteristic of α-helical structure, typical of PrP^{C}. Interestingly, the addition of HA or FA to the protein provokes a decrease in negative ellipticity (FIG. 6). In particular, the effect is stronger in presence of HA both for MoPrP(89-231) (FIG. 6A) and MoPrP(23-231) (FIG. 6C). Moreover, time-dependent transition of MoPrP(23-231) in the presence of HA was observed (FIG. 6D). Changes in molar ellipticity could be related to two hypotheses: (a) they are due to conformational changes of the secondary structure (i.e. loss of α-helical content) or (b) partial protein precipitation. To demonstrate that changes in molar ellipticity are due to the precipitation of the PrP in the presence of HSs, the authors measured adsorption of 20 μg of MoPrP(89-231) and MoPrP(23-231) in the presence of 1 μg/mL to 20 μg/mL of HA and FA. No prion protein was detected by Western blot and BCA (Pierce) analysis of the supernatant solutions after ultracentrifugation (100,000 g) at HSs concentration up to 5 μg/mL (FIG. 7). Finally, the authors measured the propensity of HSs on the binding with MoPrP(23-231) and Fc_HuPrP(23-230) using the method of competitive ELISA. For both proteins authors observed a significant decrease in absorbance due to the competitive effect of HA versus the binding site of the antibody. The authors' results suggest that HA could PrP specifically (FIG. 8).

### Example 5

### Extraction and Characterization of Humic Substances

Materials and methods—Extraction and purification of HS was carried on the basis of the procedures published by International Humic Substances Society (IHSS) and Sequi et al. (Sequi et al., 1986), both previously reported (R. S. Swift, 1996) with the following amelioration in order to optimize the analytical efficiency. Briefly, HS were extracted from 2 mm-sieved soil sample with 0.1 M NaOH (1:5 wt/vol). The suspension was left overnight under a N₂ atmosphere with constant shaking. After a slower centrifugation at 13,000 rpm to remove the bulky material, the extract was centrifuged at 24,000 rpm and filtrated through a 0.45 μm nitrocellulose filter. The filtrate was then acidified until pH 2 with H₂SO₄ to precipitate humic acids. After centrifugation the supernatant was collected, and the pellet (humic acids, HA) resuspended with 0.5 NaOH and stored. The supernatant was fed on a column packed with polyvinylpyrrolidone (PVP), previously equilibrated in 0.01 M H₂SO₄. The eluate (the non-retained, non-humified fraction) was discarded, while the brown-coloured retained fraction (fulvic acids, FA), was subsequently eluted with 0.5 M NaOH. Both fractions were passed through H+ exchanging resin to remove metal ions and adjusted to pH 7. The organic carbon content of the HA and FA fraction were measured by wet oxidation method (It. Min. Lex n.248 Oct. 21^{st} 1999).

In conclusion, the present invention surprisingly demonstrate that non-cytotoxic concentrations of naturally occurring humic (HA) and fulvic (FA) substances can rapidly eliminate PrP^{Sc} from chronically infected ScGT1 cells.

Furthermore, the amyloid seeding assay (ASA) of MoPrP(89-231) and MoPrP(23-231) showed a considerably longer lag phase in the presence of increasing concentration of HAs and FAs.

Moreover, the interaction between recMoPrP_{C} and HA, FA using Far UV Circular Dichroism and ELISA assays is shown.

### REFERENCES

Adjou, K T, et al., J Gen Virol 84 (2003), 2595-2603.
Ar\check{c}on I., et al., 2006 Environmental Chemistry Letters 4, 191-194.
Bertsch, U., K. F. Winklhofer, et al. (2005). J Virol 79(12): 7785-91.
Borges, F., Guimarães, C., Lima, J. L. F. C., Pinto, I., Reis, S., 2005. Talanta 66, 670-673.
Campana, V., L. Zentilin, et al. (2009). Biochem J 418(3): 507-15.
Campitelli, P. A., Velasco, M. I., Ceppi, S. B., 2006. Talanta 69, 1234-1239.
Caspi, S., et al., J. Biol. Chem. 273 (1998), pp. 3484-3489.
Chien, Y. Y., Bleam, W. F., 1997. Langmuir 13, 5283-5288.
Cozzolino, A., et al., 2001. Soil Biology and Biochemistry 33(4-5), 563-571.
Daude, N., M. Marella, et al. (2003). J Cell Sci 116(Pt 13): 2775-9.
De Paolis, F., Kukkonen, J., 1997. Chemosphere 34, 1693-1704.
Doh-ura K, Iwaki T, Caughey B. J. Virol. 2000; 74:4894-4897.
Evans D. A., et al., (1989) J. Am. Med. Assoc. 10, 2551-2556.
Fang, F., et al., 1998. Analytica Chimica Acta 373(2-3), 139-151.
Fetsch, D., Havel, J., 1998. Journal of Chromatography A 802(1), 189-202.
Genovesi S., et al., 2007 PLoS ONE 2 (10): 1-6 cod e1099.
Gevao, B., Semple, K. T., Jones, K. C. 2000. Environmental Pollution 108, 3-14.
Guetzloff, T. F., Rice, J. A., 1994. The Science of the Total Environment, 152, 31-35.
Hebert L. E., et al., (2003) Arch Neurol 60, 1119-1122.
Ishiwata, S., Kamiya, M., 1999. Chemosphere 39, 1595-1600.
Katzman R. (1986) N. Engl. J. Med. 314, 964-973.
Klaus, U., et al., 2000. Environmental Science and Technology 34, 3514-3520.
Klöcking, R. et al., 1972. Experientia, 28(5), 607-608.
Klöcking, H-P., 1991. In: Humic substances in the aquatic and terrestrial environment, Vol. 33 of lecture notes in Earth Sciences (Bhattacharji, S., Friedmann, G. M., Neugebauer, H. J., Seilacher, A., Eds.), Springer-Verlag, Berlin, pp. 423-428.
Kocisko, D. A., G. S. Baron, et al. (2003). J Virol 77(19): 10288-94.
Kurk, D. N., Choppin, G. R, 2000. Radiochimica Acta 88(9-11), 583-586.
Kuwata, K., N. Nishida, et al. (2007). Proc Natl Acad Sci USA 104(29): 11921-6.
Lee, R. T., et al. 2001. Chemosphere 43(8), 1063-1070.
Leita, L., et al., 2001. Soil & Sediment Contamination: an International Journal, 10, 483-496.
Leita L., et al., 2006 Soil Biology and Biochemistry. Vol 38: 1638-1644.
Leita, L., et al., 2009. Environmental Pollution 157(6), 1862-1866.
Lilienfield D. E. (1993) in Neurodegenerative Diseases (Calne D. B., ed.), pp 399-425. W.
B. Saunders, New York.
Loya, S., et al., 1993. Journal of Natural Products 52(12), 2120-2125.
Lubal, P., et al., 1998. Talanta 47, 401-412.
Lubal, P., et al., 2000. Talanta 51(5), 977-991.
Myneni, S. C. B., 2002. Science 295, 1039-1041.
Nam, K., Kim, J. Y., 2002. Environmental Pollution 118, 427-433.
Pacheco, M. L., Havel, J., 2001. J. of Radioanalytical and Nuclear Chemistry 248, 565-570.
Pacheco, M. L., Pena-Mendez, E. M., Havel J., 2003. Chemosphere 51(2), 95-108.
Peña-Méndez, E. N., Havel, J., Pataka, J., 2005. J. of Applied Biomedicine 3, 13-24.
Piccolo, A., 2002. Advances in Agronomy 75, 57-134.
Priola S A; Raines A; Caughey W S Science (New York, N.Y.) 2000; 287(5457):1503-6.
Prusiner S. B. (1998) Prions (Les Prix Nobel Lecture), in Les Prix Nobel (Frangsmyr T., ed.), pp 268-323. Almqvist & Wiksell International, Stockholm, Sweden.
Riede, U. N., et al., 1991. Virchows Arch B Cell Pathol Incl Mol Pathol 60(1), 27-34.
Schatzl, H. M., L. Laszlo, et al. (1997). J Virol 71(11): 8821-31.
Schiller, F., et al., 1979. Dermatoi Monatsschr 165(7), 505-509.
Schmitt, Ph., et al., 1997. Chemosphere 35, 55-75.
Schneider, J., et al., 1996. Virology 218(2), 389-395.
Schulten, H.-R., Thomsen, M., Carlsen, L., 2001. Chemosphere 45, 357-369.
Schols, D., et al., 1991. J. of Acquired Immune Deficiency Syndromes 4(7), 677-685.
Shermer, C. L., et al., 1998. Journal of the science of food and agriculture 77(4), 479-486.
Sigurdsson and Wisniewski, Exp Rev Vaccines 4 (2005), pp. 607-610.
Spencer B., et al. (2007) Expert Opin Biol Ther 7, 1853-1867. Steed, J. W., Atwood, J. L., 2000. Supramolecular Chemistry. John Wiley & Sons, London.
Stevenson F. J., 1994. Humus Chemistry: Genesis, Composition, Reactions. John Wilwy & Sons, New York.
Supattapone, S. et al., 1999. PNAS, 25: 14529-14534.
Von Wandruszka R, 1998. Soil Sci 163, 12, 921-930.
Wisniewski and Sigurdsson 2007 T., FEBS J 274 (2007), pp. 3784-3798.

1. A humic substance for use in the treatment of a neurodegenerative disease.

2. The humic substance according to claim 1 wherein the neurodegenerative disease is selected from the group consisting of: prion disease, Alzheimer's disease, Parkinson's disease, amyotrophic lateral sclerosis and Huntington's disease.

3. The humic substance according to claim 2 wherein the prion disease is selected from the group consisting of: scrapie, bovine spongiform encephalopathy, chronic wasting disease and Creutzfeldt-Jakob disease.

4. The humic substance according to claim 1 wherein the humic substance is humic acid, fulvic acid or a mixture thereof.

5. A composition comprising a humic substance and appropriated diluents or excipients for use in the treatment of a neurodegenerative disease.

6. The composition according to claim 5 wherein the neurodegenerative disease is selected from the group consisting of: prion disease, Alzheimer's disease, Parkinson's disease, amyotrophic lateral sclerosis and Huntington's disease.

7. The composition according to claim 6 wherein the prion disease is selected from the group consisting of: scrapie, bovine spongiform encephalopathy, chronic wasting disease and Creutzfeldt-Jakob disease.

8. The composition according to claim 5 wherein the humic substance is humic acid, fulvic acid or a mixture thereof.

9. A method for the treatment of a neurodegenerative disease comprising administering to a subject in need thereof a humic substance.

10. The method according to claim 9 wherein the neurodegenerative disease is selected from the group consisting of: prion disease, Alzheimer's disease, Parkinson's disease, amyotrophic lateral sclerosis and Huntington's disease.

11. The method according to claim 10 wherein the prion disease is selected from the group consisting of: scrapie, bovine spongiform encephalopathy, chronic wasting disease and Creutzfeldt-Jakob disease.

12. The method according to claim 9 wherein the humic substance is humic acid, fulvic acid or a mixture thereof.
